SEQUENCE LISTING (2 sequences)

SEQ ID NO: 1 - DNA, 759 bp, Pythium oligandrum
  ITS rRNA gene for strain M1
atcattaccacacctaaaaactttccacgtgaaccgttataactatgttctgtgcttcgtcgcaagacttgaggctgaacgaaggtgagtctgcgtctattttggatgcggatttgctgatgttattttaaacacctattacttaatactgaactatactccgaatacgaaagtttttggttttaacaattaacaactttcagcagtggatgtctaggctcgcacatcgatgaagaacgctgcgaactgcgatacgtaatgcgaattgcagaattcagtgagtcatcgaaattttgaacgcatattgcactttcgggttatgcctggaagtatgcctgtatcagtgtccgtacatcaaacttgcctttctttttttgtgtagtcaaaattagagatggcagaatgtgaggtgtctcgcgctgtctttttaaagatggttcgagtccctttaaatgtacgttgattctttcttgtgtctgcgaattgcgatgctatgctctttgtgatcggtttagattgctttgcgctggtgggcgacttcggttaggacatatggaagcaacctcaattggcggtatgttcggctttgcctgacgttaagctaagcgagtgtagttttctgtcttttccttgaggtgtacctgtcgtgtgtgaggttgatttaggctatatggttgcttggttgtgtggtttagcgttttcagacgcctgcttcggtaggtaaaggagacaacaccaatttgggactgagagtttact

SEQ ID NO: 2 - DNA, 683 bp, Pythium oligandrum
  Mitochondrial COXII cytochrome oxidase for strain M1
atggaaggtattattaactttcatcatgatttagtattttttttaattattgtgactgtttttgtttgttggttattatttagagtaatcgtattattcgatgaaaaaaaaaacccaatacctgctacatttgtacatggagcaactattgaaattatttggacaacaattccagcattaattttattaaccgtagcagttccatcttttgctttattatattcaatggatgaaattattgatccaattataactttaaaagtaataggtagtcaatggtactggagttatgaatattctgataatttagaatttgcagatgaacctttaatttttgatagttacatggttcaagataatgacttagaaataggacaatttaggttattagaagtagacaaccgtgttgttgtaccaactaatagccatattagagttttaataacagcttctgacgttttacattcatgggctataccctctttaggtttaaaattagatgcttgtccaggtcgtttaaatcaaacttcaatgtttattaaaagagaaggtgtattttacggtcaatgtagtgaaatatgtggtataaatcatggttttatgccaatagttgttgaagcagtttcattagaagattatttagtttggttaaaaaacaaattaattttgattttaatgtataa

(19) United States
(12) Patent Application Publication
(10) Pub. No.: US 2021/0386073 A1
(43) Pub. Date: Dec. 16, 2021

(54) BIOLOGICAL ANTIFUGAL LIQUID PREPARATION WITH MICROORGANISM PYTHIUM OLIGANDRUM AND METHOD OF PRODUCTION

(71) Applicant: BIOPREPARÁTY, SPOL. S R.O, Únetice (CZ)

(72) Inventors: Martin Suchánek, Praha 6 (CZ); Jan Moravec, Statenice (CZ); Tomás Vanek, Praha 5 (CZ); Adam Stípek, Louny (CZ)

(21) Appl. No.: 16/326,032

(22) PCT Filed: Oct. 16, 2017
(86) PCT No.: PCT/CZ2017/000064

§ 371 Date: Feb. 15, 2019

(30) Foreign Application Priority Data
Oct. 14, 2016 (CZ) PV 2016-645
Oct. 16, 2017 (CZ) PV 2017-661

(51) Int. Cl. A01N 65/03

(52) U.S. Cl. CPC A01N 65/03

(57) ABSTRACT

The liquid biological antifungal product containing the Pythium oligandrum microorganism containing a stabilized suspension of the Pythium oligandrum microorganism contains 0.05 to 10.0% weight culturing biomass of the Pythium oligandrum microorganism with content of cultivation medium, cell forms of this microorganism and substances produced by this microorganism and 90.0 to 99.95% weight stabilizer, where-by the pre-determined number of dormant oospores in 1 ml of this liquid biological anti-fungal product is, following normal standardization, between 1×10³ and 2×10⁷. The liquid biological antifungal product containing the Pythium oligandrum microorganism containing a stabilized suspension of the Pythium oligandrum microorganism contains 0.05 to 10.0% weight culturing biomass of the Pythium oligandrum microorganism with content of cultivation medium, cell forms of this microorganism and substances produced by this microorganism; and 79.77 to 99.95% weight stabilizer, and the remainder, up to 100% weight, a minimum of one modifying/application substance from a group including filling, aroma and vitamin; whereby the pre-determined number of dormant oospores in 1 ml of this liquid biological antifungal product is, following normal standardization, between 2.5×10⁴ and 1.0×10⁶. The Pythium oligandrum microorganism is the Pythium oligandrum Dreschler ATTC 38472 strain, which was deposited in the Czech Collection of Microorganisms (CCM) at Masaryk University in Brno under the appellation of Pythium oligandrum M1. The stabilizer may be water, a salt solution, oil or a concentrated solution of osmolytes. The method of production of this liquid biological antifungal product is claimed.

### TECHNICAL FIELD

The invention concerns a liquid biological antifungal product containing the Pythium oligandrum microorganism.

The invention also concerns the method of producing this product.

### BACKGROUND OF THE INVENTION

Chemical antifungal products have in recent decades aroused rising fears from the perspective of their negative influence on the environment, and indeed the user. Nonetheless, it can be said that the replacement of these with effective biological products based on microbes that are able to eliminate molds in a targeted and considerate way has not proceeded to the desired extent. Only 14 microbial types are currently registered for this purpose in the countries of the EU, this in spite of very intensive research, as stressed in a recent article by Gerbore et al from 2014 [1].

The practical application of a biological product which uses the antifungal effects of the Pythium oligandrum microorganism has been known since the 1970s, in that we can here refer to the classic works of Dreschler [2] from 1943 and Deacon [3] from 1976 dedicated to the biology of this microorganism and describing its mycoparasitic properties. It is from that time that we have patent U.S. Pat. No. 4,259,317 [4], authors Veselý and Hejdánek, priority date 5 Jul. 1978, dedicated to dry powder products containing oospores of Pythium oligandrum Dreschler, for protection against mold-related diseases emanating from sugar beet shoots having an content of oospores of a minimum 1 million oospores per gram of product, whereby a quantity of between 50 and 500 million oospores per gram of product is stated as the optimum level. As an applicable alternative to dry powder products, the authors of this invention accept a liquid suspension of oospores, but specify a number of disadvantages for the application which they prepared, in particular the instability of such products, as limiting their practical application to around 2 weeks. After a longer period of time, there is an unacceptable reduction in the emergence of oospores, which is essential for application of the mycoparasitic protection mechanism, in consequence of autolysis or contamination by hyperparasites. As far as the source of individual strains of Pythium oligandrum is concerned, authors Baker and Lifshitz pointed in their patent U.S. Pat. No. 4,574,083 [5] to the possibility of obtaining regionally suitable strains through isolation from the soil according to the standard published method. This procedure has since been vindicated and strains from different geographical locations are now available in public collections. The following biological criteria were mainly applied at that time to identify isolates: speed of growth, optimum growth temperature—usually around 30° C., and morphology of individual reproductive forms of the sexual and asexual cycle. From this perspective, the patent which followed can be deemed ground-breaking; in this, inventor Martin, U.S. Pat. No. 5,961,971 [6], introduces the molecular characterization of individual isolates based on the precise sequential protocol of critical genes. Individual formulations and methods of applying the Pythium oligandrum microorganism in field conditions are presented in detail in the same patent document, whereby liquid formulations following growth on cheap media based on industrial waste materials and powder formulations prepared by the encapsulation of biomass dried after fermentation to become alginates or in the form of a dried and ground product are mentioned as the preferred formulations. Formulations in the form of wettable powders, standard powders. emulsified oils and granules are among the other possible formulations. Patent CZ 303 908 and the corresponding EP 2 503 892 [7], BIOPREPARÁTY, spot. s.r.o., from 2009, focuses on the post-harvest protection of crops with the use of the Pythium oligandrum microorganism. In the technological section, it concentrates on the culturing of this microorganism in standard fermenters on liquid media with cereal content, without the addition of other nutrients. The possibility is accentuated of obtaining a pure concentrate of oospores without the presence of exhausted media or the remains of mycelium, which are removed during drying in a spray drying oven at the late stages of the production process. Knowledge of the preparation of oospores of organisms other than Pythium oligandrum has been published to a limited extent. In patent application CN 102 807 957A [8], Liu X. et al, from 2012, the preparation of emerging oospores of the Phytophthora capsici microorganism is described in order to monitor resistance to various drugs and for genetic research. Liquid products for the biological protection of plants are specified in patent application US 2014/0212387 [9], Liith, from 2014, although these are exclusively liquid products that use the polyethers of modified trisiloxanes as solvents.

The preparation and stabilization of oospores and other reproductive forms of microscopic Pythium oligandrum oomycetes is also discussed to a certain extent in non-patent literature. In 1990, McQuilken et al [10] published a simple and reproducible method for the preparation of oospores in a liquid culture with the use of molasses as an economically affordable substrate. However, the issue of the ideal method of maintaining prepared oospores has not been satisfactorily resolved in either patent or non-patent literature. In 1992, McQuilken et al [11] observed the influence of different osmotics, such as glycerol, NaCl and KCl, on the speed of radial extension of hyphae and the emergence of oospores of the Pythium oligandrum oomycete by way of a comparison with the osmotic pressure presented by a soil extract at −0.5 MPa. The addition of osmolytes reduced the speed of radial extension, but had no influence on emergence to a value of around −2.0 MPa. The authors conclude that good effectiveness of the applied oospores can be expected under normally-occurring osmotic conditions in the places of application on the condition that conditions are met from the perspective of optimum temperature, generally 30° C., and acidity of pH 6.0 to 7.5. From the perspective of the storability of the oospores obtained, their high effectiveness for a period of up to 379 days when maintaining a normally prepared suspension in distilled water in the refrigerator at 4° C. is mentioned by authors Takenaka and Ishikawa [12] of 2013.

According to experts, the main obstacle to greater spread of biological defense products is quite clearly the dependence of effectiveness on specific conditions of application and practical problems with maintaining and preparing biological products to protect plants in everyday agricultural work. For this reason, resolving practical problems associated with the simple and effective storage and application of Pythium oligandrum oospores is crucial to the commercial success of biological products. In order to resolve this problem, it would be desirable to develop means of biological protection in liquid state, with high long-term stability even under standard storage conditions. However, the solutions published so far suffer from a number of disadvantages that have not been successfully eliminated even after years of intensive effort. The majority of procedures involved in the laboratory preparation of biological preparations to protect plants are complicated and only manageable at laboratory level, which significantly restricts the use of such procedures for industrial production. The preferred method of application until now, in the form of a wettable powder, which was applied in products manufactured by Czech biotechnological company Biopreparáty, spol s.r.o. (Polyversum®, Polygandron, BIOREPEL®) and sold worldwide, is not without its own complications from the perspective of targeting defensive intervention and protecting the users. Until now, there has been no simple, well-tested and consistently-defined procedure published for a formulation with sufficient storability of prepared reproductive forms of the Pythium oligandrum microorganism, or of oospores, in a simple form that can be used easily in industry.

### SUMMARY OF THE INVENTION

The specified disadvantages of the biological antifungal products applied until now are eliminated or restricted under this invention, whose essence is a liquid biological antifungal product containing the Pythium oligandrum microorganism according to independent claim 1, including a stabilized suspension of Pythium oligandrum, and containing 0.05 to 10.0% weight culturing biomass of the Pythium oligandrum microorganism with content of cultivation medium, cell forms of this microorganism and substances produced by this microorganism; and 90.0 to 99.95% weight stabilizer; whereby the number of dormant oospores in 1 ml of this liquid biological antifungal product required in advance ranges from 1×10³ to 2×10′ following normal standardization.

The essence of the liquid biological antifungal product containing the Pythium oligandrum microorganism according to the 2^{nd} independent claim of this invention is based on the fact that it includes a stabilized suspension of Pythium oligandrum and contains: 0.05 to 10.0% weight culturing biomass of the Pythium oligandrum microorganism with content of cultivation medium, cell forms of this microorganism and substances produced by this microorganism; 79.77 to 99.95% weight stabilizer; the remainder, to 100% weight, at least one modifying/application substance from a group including filling, aroma, vitamin E; whereby the number of dormant oospores in 1 ml of this liquid biological antifungal product required in advance ranges from 2.5×10⁴ to 1.0×10⁶ following normal standardization.

Preferable is the Pythium oligandrum Dreschler ATTC 38472 strain of the Pythium oligandrum microorganism, which was deposited in the Czech Collection of Microorganisms (CCM) at Masaryk University in Brno under the appellation of Pythium oligandrum M1.

The main advantage of this invention is a new liquid biological antifungal product that is suitable for long-term usage and achievement of its long-term and significant stabilization effect, whereby the stabilizers used are affordable and provide a further advantage in protecting the contained biomass of the Pythium oligandrum microorganism from microbial contamination. The claimed content of dormant cells of the microorganism specified above in the form of oospores provides a practically tested and economically affordable scope, which facilitates simple dosing for a wide range of required applications. It can use different types of biomass, processed using a suitable preparation. The lower concentration of dormant oospores, for example in tens or hundreds of dormant oospores, also brings results, although the effectiveness of this product is lower. The pre-determined number of dormant oospores can be regulated, if required, by normal standardization, i.e. by diluting or concentrating to the required value.

It is advantageous when the stabilizer contains at least one component from a group including water, salt solution, oil or solutions of osmolytes, which might be polyol solution, of saccharide or saccharide alcohol type, or salt solutions. Polyol solutions may be chosen from a group containing metabolizable or unmetabolizable solutions of various oligosaccharides containing a maximum of 10 saccharide units and a minimum of 2 saccharide units, such as maltodecaose or maltononaose or maltooctaose or maltohelpaose or maltohexaose or maltopentaose or stachyose or rafinose or sucrose or sucralose, or branched saccharides may be used.

In the ideal execution, the stabilizer is water in a quantity of, for example, 30.0 to 99.9% weight, or salt solution in a quantity of, for example, 99.9% weight, or an osmolyte in the form of sucrose in a quantity of, for example, 60.0 to 64.95% weight, or at least one oil selected from a group including paraffin oil, mineral oil, glycerol, sunflower oil, in a quantity of, for example. 79.77 to 99.9% weight.

The liquid biological antifungal product may contain other modifying or application substances for a specific area of use, such as a slow-release biodegradable matrix such as a carrier of oospores of the Pythium oligandrum microorganism, for example polyvinyl alcohol, in a quantity, for example, of 16.0% weight; or filling based on silicon oxide in a quantity of, for example, 16.0% weight; or other application/modifying substances, such as sage or mint aroma in a quantity of, for example, 0.96% weight, or vitamin E in a quantity of, for example, 0.4% weight.

The use of such application/modifying substances depends on the application of the biological antifungal product.

Consequently, a stabilized suspension of oospores of the Pythium oligandrum microorganism might use a wide range of stabilizing agents of a hydrophilic or hydrophobic nature, such as common water or oil or solutions of different affordable osmolytes.

Osmolytes can be perfectly adapted, through their chemical nature, to the required applications, since it has been shown that osmolytes might be of an ion nature, such as solutions of different salts, or of a non-ion nature, such as polyol solutions. The fact that polyols might be represented by, for example, metabolizable or unmetabolizable solutions of different defined oligosaccharides is beneficial for a number of applications, because mixtures of suitable natural or synthetic substances of the required properties can be used for this purpose. A commonly available substance such as sucrose can also be used to our advantage, but if required even commonly available unmetabolizable analogues can be used. The composition of liquid suspensions is compatible with a wide range of other substances which modify their utility properties or their applications in the form of antioxidant substances, natural activators and vitamins, for example silicon oxide as a filling, vitamin E acetate and other substances.

It was discovered, somewhat surprisingly, that if the cell percentage produced by solid or liquid culturing following the end of preparation is put in the form of a suspension, or concentrated suspension, the reproductive forms of the Pythium oligandrum microorganism retain the original emergence in such a suspension for at least six months if normally stored at laboratory temperature. This result becomes even more prominent when we consider the irreproducible and variable results presented in the published literature. It is stated in literature, for example, that a suspension of oospores prepared in distilled water and stored for a period of 188 or 384 days retained the original emergence when stored in a refrigerator at 4° C. Nonetheless, the entirely absent description of the method of preparing such described preparations makes it almost impossible to evaluate their scientific validity, not to mention practical applicability, when a storage temperature of 4° C. might not even be available under working conditions on farms.

The liquid biological antifungal product containing the Pythium oligandrum microorganism is obtained using the method of production according to this invention, the essence of which lies in the fact that a liquid medium containing extract of cereals, cane molasses and other essential nutrients is sterilized in a steam sterilizer by way of the aerobic culturing of the Pythium oligandrum oomycete in liquid phase. After cooling, it is inoculated with one of the selected strains of Pythium oligandrum. The biomass is taken and processed after the end of culturing, which takes several days, preferentially 13. After the end of the culturing stage of Pythium oligandrum in liquid phase, the biomass is homogenized with the liquid medium so that a minimum of 95% of the particles in the suspension are between 0.050 and 0.300 mm, preferentially 0.125 mm. The homogeneous suspension characterized by the number of oospores is concentrated or diluted according to the number of oospores in the solution to the pre-determined concentration of oospores in the biological antifungal product. The suspension is preferentially filtered after the stage of homogenization and concentration or dilution.

An aqueous suspension is stabilized during homogenization with an additive of osmolyte and is stored in large, sterile containers at temperatures of lower than 8° C.

In the case of an anhydrous suspension of the Pythium oligandrum microorganism, the material obtained after the end of the culturing stage on a solid substrate or at the liquid phase is homogenized until reaching a value of a minimum 95% of the particles in the suspension between 0.050 and 0.300 mm, preferentially 0.125 mm. The obtained homogenous suspension characterized by the number of oospores is centrifuged. After the removal of supernatant, the centrifuged material is supplemented with oil to achieve the pre-determined concentration of oospores for the production of the biological antifungal product in such a quantity that the resulting concentration of oospores in the biological antifungal product is correct to the desired concentration and the material obtained in this way is subsequently re-suspended by homogenization.

The resolution presented would appear to be suitable even from the perspective of significant simplification of the biotechnological process of preparing the Pythium oligandrum microorganism and thus the significant reduction in running costs for the manufacturers of such products. |Filtration or separation of the cultivated biomass from the production medium, its drying and subsequent grinding to the required size of particles entails several processes that are relatively demanding on energy and technology, difficult to control and susceptible to the possibility of contamination, which could, as an end result, significantly damage the resulting product, if not carried out under precisely-defined and industrially demanding conditions. By contrast, the technologically simple suspension of the biomass obtained in dense and viscose solutions of osmolytes results in a very stable product that is immediately suitable for a range of practical applications. From the practical perspective, it is a significant advantage that the prepared suspension of the Pythium oligandrum microorganism can be transferred from culture dishes or fermenters almost without waste, which minimizes the costs of their cleaning and maintenance and also minimizes the possibility of product leaking into the work environment of the production premises. Moreover, the very fact that the product obtained in this way is stable over the long-term, even during storage at normal temperatures, also significantly reduces running costs and makes it easier to handle the product.

It was also discovered, surprisingly, that apart from the stabilizing effect of concentrated solutions of osmolytes, the conserving effect of such solutions, which is well-known from production and common practice, is significant in the specific case of the suspensions of oospores of the microscopic oomycete Pythium oligandrum which we examined. A product stored in this way is therefore protected from contamination by undesirable microorganisms, which, even in the very unlikely case of penetration of such stabilized suspensions, are unable to reproduce in the proposed chemical environment and cannot therefore contaminate the end product. This advantage is fundamental in current production practice, particularly in light of the ever stricter requirements on the level of accidental contamination in pesticides, biocides and cosmetic products.

In addition to the role of stabilizer and the role of conserving substance, osmolyte solutions also play the role of adhesive, a fact which can be practically applied in many applications, whether in the form of aqueous solutions after the dilution of stabilized suspensions of oospores to the optimum concentration with application by soaking or for application by gentle dispersion of microscopic particles (so-called misting). In all of the examples of application specified above, polyol solutions therefore aid effective adhesion and subsequent even application to the treated areas, plants or crops.

Last, but not least, it must be mentioned that in the case of using metabolizable osmolytes, these substances might serve as a nutrient which is important during colonization and settlement of the target places directly at the places of application, whereby this is a significant nutrient particularly during the first stage connected with the transfer of oospores to myceliar form. They can subsequently take on the role of nutrients important for the long-term growth and preservation of the microscopic oomycete Pythium oligandruni in the place of application of a nutrient of more complex nature, represented by imperfectly fermented substrate used during biotechnological growth and reproduction of oospores during biotechnological culturing.

The specific content of oospores and other reproductive forms of microscopic oomycetes in suspensions according to the resolution presented is comparable with or higher than previously-tested and sold loose products, which is important from the perspective of the logistics and economics of distribution of a product made in this way. However, the new formulation also makes it possible to use lower concentrations in comparison with the previous resolution.

We have actually seen a significant reduction in composition complexity in the new liquid biological antifungal product in connection with the technological procedure used, without this being linked to any limitation in the function or effectiveness of the liquid product formulated in this way. As far as the cell and particle composition of the newly-proposed product is concerned, this almost entirely entails stabilized oospores with a very low percentage of mycelial fragments, ground millet and other components. These components obviously do not sediment in the dense environment created by concentrated osmoles, which surprisingly guarantees good homogeneity of the product, incomparably better than in the case of wettable powders applied in the form of aqueous suspensions, when the preparation was very heterogeneous from the perspective of the size of the particles therein. This particularly applies to the particles of silicon oxide contained in the products made until now; by contrast, silicon oxide is absent in most cases in the resolution according to the submitted invention (with the exception of toothpaste, where it is used as an abrasive). Consequently, from the perspective of the needs of practical applications, there is no clogging of filters and other technological equipment during the application of the product.

If the presence of a lower quantity of metabolizable osmolytes is detrimental to a certain application, these can easily be replaced with chemically similar unmetabolizable osmolytes without affecting in any way the most important properties of the product.

If it is more appropriate from the technological perspective to dry the product obtained, freeze-drying (lyophilization) would appear to be suitable here, in apparatus with a high vacuum value, since the use of concentrated solutions of osmolytes as cryopreservatives and protective substances during lyophilization is well-known and widely-used. By way of example, we refer to the osmolytes widely used as protective cryopreservatives in a number of sensitive applications, including the cryopreservation of human oocytes described. for example, in an article by Wright [15] from 2004.

A considerable advantage of the products obtained according to the submitted invention is that such products are suitable for all applications in which it would appear beneficial to use the anti-mold, mycoparasitic, elicitor and growth properties of the Pythium oligandrum microorganism. In particular, therefore, the use of this product as a plant biofungicide, for human and veterinary use of the Pythium oligandrum microorganism, for the destruction of biofilms and the removal of dysbiosis occurring in the case of various medical diagnoses and also for civic protection from molds occurring in the living and/or working environment. Depending on the matrices used in the liquid suspension concentrate, it can be used as a protective spray of plants in the presence of a degradable biopolymer, which acts as a protective factor and as a gradually degradable matrix, meaning as a “carrier” of Polyversum material with oospores of the active microorganism Pythium oligandrum.

Protection against molds, funguses, pathogenic bacteria and yeasts with the use of a stabilized, concentrated suspension of oospores of the Pythium oligandrum microorganism, consisting of the application of such stabilized, concentrated suspension, in undiluted or diluted form, to an area affected by mold or fungus or pathogenic bacteria or pathogenic yeasts or microbial dysbiosis, makes it possible to define a wide scope of applications for the stabilized liquid concentrate.

In the sphere of plant protection, the composition can be used to provide protection against molds, funguses or pathogenic bacteria and yeasts such that the stabilized, concentrated suspension, in diluted or undiluted form, is applied to plants or their surroundings or seeds or crops or fruits either before harvest or after harvest, for example in warehouses; this includes application in the form of aerosols, self-soluble capsules containing concentrated suspension, additional fertilization at the bottom of the plant, soaking crops, spraying crops, seed treatment, misting crops, fertilizing and additional fertilizing as a component part of comprehensive fertilizers and application in the form of hydroponics.

When protecting the oral cavity, the stabilized, concentrated suspension in diluted or undiluted form can be applied to a place affected by oral plaque or periodontal disease, the occurrence of infected, non-healing wounds, in particular among diabetics, the occurrence of dermatophytal or yeast-related infectious diseases and the occurrence of other symptoms connected with microbial dysbiosis of the skin and human surface membranes.

For use in civic protection, the stabilized, concentrated suspension, in diluted or undiluted form, can be applied to places on walls and in masonry affected by the occurrence of fungus. Other innovative areas of use include antifungal protection of cooling equipment, air-conditioning, areas affected by flooding and deluge and other places with frequent occurrence of funguses targeted by the Pythium oligandrum microorganism.

### BRIEF DESCRIPTION OF THE DRAWINGS

The invention is further described in detail in exemplary embodiments and explained in more detail in the appended schematic drawings.

FIG. 1A, 1B, 1C elucidate in more detail a comparison of the speed of growth of the phytopathogenic fungus Fusarium graminearum without the presence of the Pythium oligandrum microorganism and when that microorganism is present.

They specifically show:

FIG. 1A the growth of a colony of the phytopathogenic fungus Fusarium graminearum on its own (square) and with simultaneous inoculation by Pythium oligandrum from dormant oospores and from liquid and solid culturing (stars and triangles);

FIG. 1B photograph of Petri dishes on which the Pythium oligandrum microorganism from dormant oospores has been inoculated on the left-hand side and the phytopathogenic fungus Fusarium graminearum (upper row) on the right-hand side and on control dishes only Fusarium graminearum fungus was inoculated, in that growth was recorded after 4 days;

FIG. 1C photograph of Petri dishes with simultaneous inoculation microorganism of Pythium oligandrum from dormant oospores of the phytopatogenic fungus Fusarium graminearum, where growth was recorded after 11 days.

FIG. 2A, 2B, 2C show the results of laboratory experiments of the growth of wheat in the presence of the phytopathogenic fungus Fusarium graminearum, the Pythium oligandrum microorganism and a combination of these two microorganisms.

They specifically show:

FIG. 2A the Erlenmeyer flasks in which wheat grew on agar soil in the presence of only the phytopathogenic fungus Fusarium graminearum, the little white spots, recorded after 20 days of inoculation at the shoot of the wheat;

FIG. 2B the Erlenmeyer flasks in which wheat grew on agar soil in the presence of both the phytopathogenic fungus Fusarium graminearum and the Pythium oligandrum microorganism; the flasks were recorded after 20 days of simultaneous inoculation at the shoot of the wheat; and

FIG. 2C the Erlenmeyer flasks in which wheat grew on agar soil in the presence of only the Pythium oligandrum microorganism; the flasks were photographed after 20 days of inoculation of this fungus at the shoot of the wheat.

FIG. 3A, 3B, 3C, 3D, 3E show the application of an antifungal product for plant protection according to exemplary embodiment 1 and subsequent analysis of the population dynamic of the Pythium oligandrum microorganism observed in a field experiment on wheat and the adjacent soil, including reaction to the presence of the fungus monitored by gene expression and genetic observation of the level of fungal contamination.

They specifically show:

FIG. 3A the content of the Pythium oligandrum microorganism determined by genetic test on plants (light-gray column) and in the surrounding, adjacent soil (dark-gray column) at different times following application;

FIG. 3B the development of temperatures measured using an infrared thermometer right in the field at the time of sampling (light-gray column) and a temperature record obtained from the local meteorological station (dark-gray column);

FIG. 3C the course of gene expression for cellulase (POCELL), endo- -glucanase (POENDO) and a structural protein rich in serine and threonine (POSTRU) in individual parts of the plant and in surrounding soil (So—soil, R—root, St—stem, L—leaf) observed at time 6 to 120 hours, whereby the level of gene expression was standardized to the expression of the constitutive gene -tubulin (POTUBU) and is expressed without the application of the product according to the cited methodology [12];

FIG. 3D the level of fungal contamination in identical samples as in panel C measured using quantitative PCR with general amplification primers for the amplification of fungus.

FIG. 3E a correlation graph monitoring the correlation between the level of gene expression for cellulase (POCELL) and the level of fungal contamination.

FIG. 4A, 4B, 4C, 4D show a test of the effectiveness of toothpastes according to exemplary embodiment 4 from the perspective of their ability to eliminate biofilms artificially created on ceramic hydroxylapatite plates, with the use of saliva from a healthy individual in the upper part of the figure and the use of saliva from an individual suffering from periodontal disease in the lower part of the figure.

They specifically show:

FIG. 4A a photograph of a six-hole plate with the use of saliva from a healthy individual after the release of rinsed biofilms in the solution and the removal of ceramic hydroxylapatite plates which were previously cleaned with a brush without paste, with Odol toothpaste, with Enzycal toothpaste, with paste containing glycerol according to exemplary embodiment 4 without the addition of the Pythium oligandrum microorganism, with paste containing glycerol according to exemplary embodiment 4 without the addition of silicon oxide and full paste containing glycerol according to exemplary embodiment 4;

FIG. 4B a quantitative evaluation of the content of the biofilm by measuring the intensity of coloring in individual holes of FIG. 1A on a plate spectrophotometer at a wavelength of 562 nm;

FIG. 4C a photograph of a six-hole plate with the use of saliva from an individual with periodontal disease after the release of rinsed biofilms in the solution and the removal of ceramic hydroxylapatite plates which were previously cleaned with a brush without paste, with Odol toothpaste, with Enzycal toothpaste, with paste containing glycerol according to exemplary embodiment 4 without the addition of the Pythium oligandrum microorganism, with paste containing glycerol according to exemplary embodiment 4 without the addition of silicon oxide and full paste containing glycerol according to exemplary embodiment 4; and

FIG. 4D a quantitative evaluation of the content of the biofilm by measuring the intensity of coloring in individual holes of FIG. 1C on a plate spectrophotometer at a wavelength of 562 nm.

### EXEMPLARY EMBODIMENTS OF THE INVENTION

The liquid biological antifungal product contains the Pythium oligandrum microorganism and in all exemplary embodiments the Pythium oligandrum microorganism is of the Pythium oligandrum Dreschler ATTC 38472 strain, which was deposited in the Czech Collection of Microorganisms (CCM) at Masaryk University in Brno, under appellation Pythium oligandrum M1, and it is under this appellation that it is presented hereunder in exemplary embodiments.

Below is the Sequence Protocol of this microorganism according to ST25_PCT:

| | |
|---|---|
| <160> | 2 |
| <210> | 1 |
| <211> | 759 |
| <212> | DNA |
| <213> | Pythium oligandrum strain M1 |
| <221> | ITS rRNA gene |
| <400> | 1 |

| | |
|---|---|
| atcattacca cacctaaaaa ctttccacgt gaaccgttat aactatgttc tgtgcttcgt | 60 |
| cgcaagactt gaggctgaac gaaggtgagt ctgcgtctat tttggatgcg gatttgctga | 120 |
| tgttatttta aacacctatt acttaatact gaactatact ccgaatacga aagtttttgg | 180 |
| ttttaacaat taacaacttt cagcagtgga tgtctaggct cgcacatcga tgaagaacgc | 240 |
| tgcgaactgc gatacgtaat gcgaattgca gaattcagtg agtcatcgaa attttgaacg | 300 |
| catattgcac tttcgggtta tgcctggaag tatgcctgta tcagtgtccg tacatcaaac | 360 |
| ttgcctttct ttttttgtgt agtcaaaatt agagatggca gaatgtgagg tgtctcgcgc | 420 |
| tgtcttttta aagatggttc gagtcccttt aaatgtacgt tgattctttc ttgtgtctgc | 480 |
| gaattgcgat gctatgctct ttgtgatcgg tttagattgc tttgcgctgg tgggcgactt | 540 |
| cggttaggac atatggaagc aacctcaatt ggcggtatgt tcggctttgc ctgacgttaa | 600 |
| gctaagcgag tgtagttttc tgtcttttcc ttgaggtgta cctgtcgtgt gtgaggttga | 660 |
| tttaggctat atggttgctt ggttgtgtgg tttagcgttt tcagacgcct gcttcggtag | 720 |
| gtaaaggaga caacaccaat ttgggactga gagtttact | 759 |

| | |
|---|---|
| <210> | 2 |
| <211> | 683 |
| <212> | DNA |
| <213> | Pythium oligandrum strain M1 |
| <221> | Mitochondrial COXII cytochrome oxidase |
| <400> | 2 |

| | |
|---|---|
| atggaaggta ttattaactt tcatcatgat ttagtatttt ttttaattat tgtgactgtt | 60 |
| tttgtttgtt ggttattatt tagagtaatc gtattattcg atgaaaaaaa aaacccaata | 120 |
| cctgctacat ttgtacatgg agcaactatt gaaattattt ggacaacaat tccagcatta | 180 |
| attttattaa ccgtagcagt tccatctttt gctttattat attcaatgga tgaaattatt | 240 |
| gatccaatta taactttaaa agtaataggt agtcaatggt actggagtta tgaatattct | 300 |
| gataatttag aatttgcaga tgaaccttta atttttgata gttacatggt tcaagataat | 360 |
| gacttagaaa taggacaatt taggttatta gaagtagaca accgtgttgt tgtaccaact | 420 |
| aatagccata ttagagtttt aataacagct tctgacgttt tacattcatg ggctataccc | 480 |
| tctttaggtt taaaattaga tgcttgtcca ggtcgtttaa atcaaacttc aatgtttatt | 540 |
| aaaagagaag gtgtatttta cggtcaatgt agtgaaatat gtggtataaa tcatggtttt | 600 |
| atgccaatag ttgttgaagc agtttcatta gaagattatt tagtttggtt aaaaaacaaa | 660 |
| ttaattttga ttttaatgta taa | 683 |

### Example 1

Liquid Biological Antifungal Product Intended for Spraying Crops with High Percentage by Weight of Biomass

### Preparation:

Biomass obtained through culturing of the Pythium oligandrum M1 microorganism on a solid substrate was mixed in a homogenizer vessel (industrial mixer) with demineralized water and homogenized. Homogenization proceeded at 3,000-5,000 revolutions per minute for a period of 3 minutes. Subsequently, an osmolyte (sucrose) was added to the initial, undiluted suspension in a quantity corresponding to the resultant concentration by weight of 60% weight and to the required number of oospores. The suspension with osmolyte (sucrose) was thereafter homogenized in a mixer for 1 minute at 2,000 revolutions per minute. The suspension was then stored in sterile, stainless steel tanks at a temperature of under 8° C.

It was more beneficial to prepare the liquid biological antifungal product by combining the steps of homogenization in demineralized water and subsequent addition of osmolyte (sucrose) in one, in such a way that the biomass obtained through solid culturing was homogenized in the vessel of an industrial homogenizer in an osmolyte solution (65% sucrose solution). Homogenization proceeded at 3,000-5,000 revolutions per minute for a period of 3 minutes. After determining the number of oospores in the initial suspension, this suspension was diluted with osmolyte (65% sucrose solution) in the standard way such that the required concentration of oospores in the product was achieved. The suspension was then stored in sterile, stainless steel tanks at a temperature of under 8° C.

The number of dormant oospores was determined microscopically with the use of a Sedgewick-Rafter counting chamber and stability was measured based on microscopic observation of the number of viable oospores using the plasmolytic method, in accordance with the publication by Etxeberria et al [13] from 2011. If the number of viable oospores did not fall below 90% of the initial value, the product was marked as stable.

### Composition of the Product:

| | |
|---|---|
| Sucrose as stabilizer | 60% weight |
| Water as stabilizer | 30% weight |
| Culturing biomass | 10% weight |
| Number of dormant oospores of the Pythium | 5 × 10⁵ per 1 ml |
| oligandrum microorganism | |

### Properties:

Stability: 6 months at a temperature of up to 25° C.

### Example 2

Liquid Biological Antifungal Product Intended for Spraying Crops with Low Percentage by Weight of Biomass

### Preparation:

Pythium oligandrum biomass obtained by liquid cultivation was mixed in the vessel of a homogenizer (industrial mixer) with osmolyte (sucrose) and homogenized in such a way that the required number of oospores and concentration of osmolyte (sucrose) were achieved. Homogenization proceeded at a speed of 3,000-5,000 revolutions per minute for a period of 3 minutes. The suspension was then stored in sterile, stainless steel tanks at a temperature of under 8° C. The number of dormant oospores was determined microscopically with the use of a Sedgewick-Rafter counting chamber and stability was measured based on microscopic observation of the number of viable oospores using the plasmolytic method, in accordance with the publication by Etxeberria et al [13] from 2011. If the number of viable oospores did not fall below 90% of the initial value, the product was marked as stable.

### Composition:

| | | |
|---|---|---|
| Sucrose as stabilizer | 64% | weight |
| Water as stabilizer | 35.9% | weight |
| Culturing biomass | 0.1% | weight |

| | |
|---|---|
| Number of dormant oospores of the Pythium | 5 × 10⁵ per 1 ml |
| oligandrum M1 microorganism | |

### Properties:

Stability: 6 months at a temperature of up to 25° C.

### Example 3

### Liquid Biological Antifungal Product Intended for the Seed Coating

### Preparation:

Biomass obtained through culturing of the Pythium oligandrum M1 microorganism on a solid substrate was mixed in a homogenizer vessel (industrial mixer) with demineralized water and homogenized. Homogenization proceeded at 3,000-5,000 revolutions per minute for a period of 3 minutes. Subsequently, an osmolyte (sucrose) was added to the initial, undiluted suspension in a quantity corresponding to the resultant concentration by weight of 60% weight and to the required number of oospores. The suspension with osmolyte (sucrose) was thereafter homogenized in a mixer for 1 minute.

It was more beneficial to prepare the product by combining the steps of homogenization in demineralized water and subsequent addition of osmolyte (sucrose) in one, in such a way that the biomass obtained through solid cultivation was homogenized in the vessel of an industrial homogenizer in an osmolyte solution (65% sucrose solution). Homogenization proceeded at 3,000-5,000 revolutions per minute for a period of 3 minutes. After determining the number of oospores in the initial suspension, this suspension was diluted with osmolyte (65% sucrose solution) in the standard way such that the required concentration of oospores in the product was achieved. The suspension was then stored in sterile, stainless steel tanks at a temperature of under 8° C.

The suspension obtained was then stored in sterile, stainless steel tanks at a temperature of under 8° C. The number of dormant oospores was determined microscopically with the use of a Sedgewick-Rafter counting chamber and stability was measured based on microscopic observation of the number of viable oospores using the plasmolytic method, in accordance with the publication by Etxeberria et al [13] from 2011. If the number of viable oospores did not fall below 90% of the initial value, the product was marked as stable.

Stability: 6 months at a temperature of up to 25° C.

### Composition:

| | | |
|---|---|---|
| Sucrose as stabilizer | 60% | weight |
| Water as stabilizer | 36% | weight |
| Culturing biomass | 4% | weight |

| | |
|---|---|
| Number of dormant oospores of the Pythium | 2.5 × 10⁶ per 1 ml |
| oligandrum M1 microorganism | |

### Example 4

Liquid Biological Antifungal Product as a Suspension Concentrate with Low Water Content

### Preparation:

Pythium oligandrum M1 biomass from liquid cultivation was centrifuged following harvest. The supernatant was removed after centrifuging and the centrifuged material was supplemented with paraffin oil so that the resultant number of oospores was 500,000 oospores per 1 ml of product. The mixture was thereafter homogenized in an industrial mixer for 3 minutes. The suspension was stored in sterile containers at a temperature of under 8° C. The number of dormant oospores was determined microscopically with the use of a Sedgewick-Rafter counting chamber and stability was measured based on microscopic observation of the number of viable oospores using the plasmolytic method, in accordance with the publication by Etxeberria et al [13] from 2011. If the number of viable oospores did not fall below 90% of the initial value, the product was marked as stable.

### Composition:

| | | |
|---|---|---|
| Culturing biomass | 0.1% | weight |
| Paraffin oil as stabilizer | 99.9% | weight |

| | |
|---|---|
| Number of dormant oospores of the Pythium | 5 × 10⁵ per 1 ml |
| oligandrum M1microorganism | |

### Properties:

Stability: 6 months at a temperature of up to 25° C.

### Example 5

Liquid Biological Antifungal Product Applied in the Form of Nanofibers Constituting a Slow-Release Degradable Matrix for the Pythium oligandrum M1 Microorganism

### Preparation:

Pythium oligandrum biomass from liquid cultivation was centrifuged following harvest. The supernatant was removed and the centrifuged material was supplemented with osmolyte (65% sucrose) and biopolymer, which was thereafter processed using the technology of electrostatic spinning with the use of the non-toxic, biodegradable polymer polyvinyl alcohol (PVA). The number of dormant oospores was determined microscopically with the use of a Sedgewick-Rafter counting chamber and stability was measured based on microscopic observation of the number of viable oospores using the plasmolytic method, in accordance with the publication by Etxeberria et al [13] from 2011. If the number of viable oospores did not fall below 90% of the initial value, the product was marked as stable.

### Composition:

Main ingredient (liquid suspension of Pythium oligandrum microorganism) 84% weight of which:

| | |
|---|---|
| Sucrose as stabilizer | 63% weight |
| Water as stabilizer | 36% weight |
| Culturing biomass | 1% weight |

### 2. PVA Carrier

| | |
|---|---|
| PVA as degradable carrier | 16% weight |
| Number of dormant oospores of the Pythium oligandrum | 5.10⁶ per 1 ml |
| M1 microorganism | |

### Properties:

Stability: 6 months at a temperature of up to 25° C.

### Example 6

Liquid biological antifungal product applied in the form of impregnating coating or spray Preparation:

Pythium oligandrum M1 biomass from liquid cultivation was homogenized after harvest and filtered using the filtering technique of separating particles with a size of up to 300 m. After centrifuging, this prepared biomass was mixed with mineral oil of low viscosity such that the required number of oospores in 1 ml of product was achieved. The number of dormant oospores was determined microscopically with the use of a Sedgewick-Rafter counting chamber and stability was measured based on microscopic observation of the number of viable oospores using the plasmolytic method, in accordance with the publication by Etxeberria et al [13] from 2011. If the number of viable oospores did not fall below 90% of the initial value, the product was marked as stable.

### Composition:

| | |
|---|---|
| Culturing biomass | 0.1% weight |
| Mineral oil as stabilizer | 99.9% weight |
| Number of dormant oospores of the Pythium | 2 × 10⁵ per 1 ml |
| oligandrum M1 microorganism | |

### Properties:

Stability: 6 months at a temperature of 10-45° C. (depending on the type of secondary substance).

### Example 7

### Liquid Biological Antifungal Product Applied as a Sanitation Product for Air-Conditioning

### Preparation:

Pythium oligandrum M1 biomass from liquid culturing was homogenized after harvest and filtered using the filtering technique of separating particles with a size of up to 100 m. After centrifuging, the biomass was diluted with a salt solution to the required number of oospores per 1 ml of product. The principle of application is rinsing or spraying the filters or piping of air-conditioning or cooling units. The presence of the Pythium oligandrum M1 microorganism guarantees resistance to molds and fungal diseases.

### Cleaning Pollen Filters:

The filter was submerged in a sanitary solution and was left in this for a period of 10-30 minutes, then rinsed. The alternative is to spray the same solution on the filter in its operating position. Dirt and remnants of the agent will be blown out by compressed air.

### Sanitation of Vaporizers and Filters:

After cleaning vaporizers and/or filters, a solution of the product is applied, without rinsing. An excellent sanitation effect is achieved even when the product is only used as a washing detergent. The number of dormant oospores was determined microscopically with the use of a Sedgewick-Rafter counting chamber and stability was measured based on microscopic observation of the number of viable oospores using the plasmolytic method, in accordance with the publication by Etxeberria et al [13] from 2011. If the number of viable oospores did not fall below 90% of the initial value, the product was marked as stable.

### Composition:

| | |
|---|---|
| Culturing biomass | 0.1% weight |
| Salt solution as stabilizer | 99.9% weight |
| Number of dormant oospores of the Pythium | 5 × 10⁵ per 1 ml |
| oligandrum M1 microorganism | |

### Properties:

Stability: 6 months at a temperature of 10-45° C. (depending on the type of secondary substance).

### Example 8

### Liquid Biological Antifungal Product Applied as a Sanitation Product for the Treatment of Sediments—for Mud Deposits and Flooded Areas

### Preparation:

Pythium oligandrum M1 biomass from liquid cultivation was homogenized after harvest and filtered using the filtering technique of separating particles with a size of up to 400 m. After centrifuging, the biomass was diluted using distilled or demineralized water to the required number of oospores per 1 ml of product. The principle of action is the creation of a water suspension according to the dilution specified below and application in areas affected by contaminated deposits of mud and sediment, as a consolidation and improvement procedure to improve the properties of soils and earth. The number of dormant oospores was determined microscopically with the use of a Sedgewick-Rafter counting chamber and stability was measured based on microscopic observation of the number of viable oospores using the plasmolytic method, in accordance with the publication by Etxeberria et al [13] from 2011. If the number of viable oospores did not fall below 90% of the initial value, the product was marked as stable.

### Composition:

| | |
|---|---|
| Culturing biomass | 0.1% weight |
| Distilled water as stabilizer | 99.9% weight |
| Number of dormant oospores of the Pythium | 5 × 10⁵ per 1 ml |
| oligandrum M1 microorganism | |

The applicable utility is from 250 g per hectare at a concentration of 500,000 oospores per ml.

### Properties:

Stability: 6 months at a temperature of up to 25° C.

### Example 9

Liquid Biological Antifungal Product as a Stabilized Aqueous Suspension of Oospores of Pythium oligandrum M1

### Preparation:

Pythium oligandrum M1 biomass obtained by liquid cultivation is mixed with sterile distilled water and homogenized after centrifuging (4,000 revolutions per minute, 5 minutes) and removal of the supernatant. Homogenization proceeds at a high rate of revolutions (20,000 revolutions per minute) for a period of 1 minute. Thereafter, the suspension can be filtered and concentrated as required for the purpose of achieving a suspension concentrate containing the required number of oospores of Pythium oligandrum M1 in 1 ml, without the presence of remnants of the original medium or remnants of mycelium. The suspension material obtained can be stored in sterile tanks at a temperature of between 1 and 8° C. The number of dormant oospores was determined microscopically with the use of a Sedgewick-Rafter counting chamber and stability was measured based on microscopic observation of the number of viable oospores using the plasmolytic method, in accordance with the publication by Etxeberria et al [13] from 2011. If the number of viable oospores did not fall below 90% of the initial value, the product was marked as stable.

### Composition:

| | |
|---|---|
| Sterile distilled water as stabilizer | 99.9% weight |
| Culturing biomass | 0.1% weight |
| Number of dormant oospores of the Pythium | 5 × 10⁵ per 1 ml |
| oligandrum M1 microorganism | |

### Properties:

Stability: a minimum of 6 months at a temperature of up to 25° C.

### Example 10

Liquid Biological Antifungal Product as a Stabilized Aqueous Suspension with a High Concentration of Oospores of Pythium oligandrum M1

### Preparation:

Pythium oligandrum M1 biomass obtained through liquid cultivation was homogenized in a homogenizer vessel (industrial mixer). Homogenization proceeded at 20,000 revolutions per minute for a period of 3 minutes. The suspension was then centrifuged and, after the supernatant had been removed, the biomass was concentrated to the required number of oospores in 1 ml of product by mixing with osmolyte (65% sucrose). The suspension concentrate was stored in sterile, stainless steel tanks at a temperature of under 8° C. The number of dormant oospores was determined microscopically with the use of a Sedgewick-Rafter counting chamber and stability was measured based on microscopic observation of the number of viable oospores using the plasmolytic method, in accordance with the publication by Etxeberria et al [13] from 2011. If the number of viable oospores did not fall below 90% of the initial value, the product was marked as stable.

### Composition:

| | |
|---|---|
| Sucrose as stabilizer | 60% weight |
| Water as stabilizer | 35% weight |
| Culturing biomass | 5% weight |
| Number of dormant oospores of the Pythium oligandrum | 2.10⁷ per 1 ml |
| M1 microorganism | |

### Properties:

Stability: 6 months at a temperature of up to 25° C.

### Example 11

### Biological Antifungal Product in the Form of a Paste on a Base of Glycerol

### Preparation:

1197.1 g of glycerol of pharmaceutical quality (Glycerol 99% PharmEur) was poured into a laboratory homogenizer and 239.4 g of silicon oxide Sident® 22 S, 43.1 g of culturing biomass, 14.4 g of vitamin E and 6 g of sage aroma were carefully sprinkled in. The mixture was mixed at 60 revolutions per minute for a period of 10 minutes at laboratory temperature. The mixture was thereafter put into plastic tubes with stopper, 75 g at a time, with a total of 20 such tubes being filled in this way. The number of dormant oospores was determined microscopically with the use of a Sedgewick-Rafter counting chamber and stability was measured based on microscopic observation of the number of viable oospores using the plasmolytic method, in accordance with the publication by Etxeberria et al [13] from 2011. If the number of viable oospores did not fall below 90% of the initial value, the product was marked as stable.

### Composition:

| | | |
|---|---|---|
| Glycerol 99% Pharm Eur as stabilizer | 79.77% | weight |
| SiO₂ Sident ® 22 S as filling on a base of silicon | 16.00% | weight |
| oxide | | |
| Culturing biomass Pythium oligandrum M1 | 2.87% | weight |
| Vitamin E acetate as application material | 0.96% | weight |
| FCO2 Bio 35% sage aroma as aroma | 0.40% | weight |

| | |
|---|---|
| Number of dormant oospores of the Pythium | 2.5 × 10⁴ per 1 ml |
| oligandrum M1 microorganism | |

### Properties:

Stability: a minimum of 6 months at a temperature of 25° C.

### Example 12

### Biological Antifungal Product in the Form of a Paste on a Base of Olive Oil

### Preparation:

600 g of virgin olive oil was poured into a laboratory homogenizer and 120 g of silicon oxide Sident® 22 S, 21.6 g of culturing biomass, 7.2 g of vitamin E and 3 g of mint aroma were carefully sprinkled in. The mixture was mixed at 20 revolutions per minute for a period of 10 minutes at laboratory temperature. The mixture was thereafter put into plastic tubes with stopper. 75 g at a time, with a total of 10 such tubes being filled in this way. The number of dormant oospores was determined microscopically with the use of a Sedgewick-Rafter counting chamber and stability was measured based on microscopic observation of the number of viable oospores using the plasmolytic method, in accordance with the publication by Etxeberria et al [13] from 2011.

If the number of viable oospores did not fall below 90% of the initial value, the product was marked as stable.

### Composition:

| | | |
|---|---|---|
| Refined olive oil as stabilizer | 79.77% | weight |
| SiO2 Sident ® 22 S as filling on a base of silicon | 16.00% | weight |
| oxide | | |
| Culturing biomass Pythium oligandrum M1 | 2.87% | weight |
| Vitamin E acetate as application material | 0.96% | weight |
| HNA peppermint essential oil as aroma | 0.40% | weight |

| | |
|---|---|
| Number of dormant oospores of the Pythium | 2.5 × 10⁴ per 1 ml |
| oligandrum M1 microorganism | |

### Properties:

Stability: 6 months at a temperature of 25° C.

### Example 13

### Liquid Biological Antifungal Product for the Protection of Aquatic Organisms

The product is designed for the preventative protection of fish and young fish (carp, roach, bream) from a range of fungal diseases (in particular Aspergillus spp.). The product is applied preventatively in breeding tanks. The product is also intended for the preventative protection of fish eggs from attack by a range of fungal diseases (in particular Fusarium spp., Aspergillus spp.).

### Preparation:

Pythium oligandrum M1 biomass obtained through liquid cultivation was homogenized in a homogenizer vessel (industrial mixer). Homogenization proceeded at 20,000 revolutions per minute for a period of 3 minutes. The suspension was then centrifuged and, after the supernatant had been removed, the biomass was concentrated to the required number of oospores in 1 ml of product by mixing with osmolyte (65% sucrose). The suspension was stored in sterile, stainless steel tanks at a temperature of under 8° C. The number of dormant oospores was determined microscopically with the use of a Sedgewick-Rafter counting chamber and stability was measured based on microscopic observation of the number of viable oospores using the plasmolytic method, in accordance with the publication by Etxeberria et al [13] from 2011. If the number of viable oospores did not fall below 90% of the initial value, the product was marked as stable.

### Composition:

| | |
|---|---|
| Sucrose as stabilizer | 63% weight |
| Water as stabilizer | 36% weight |
| Culturing biomass | 1% weight |
| Number of dormant oospores of the Pythium oligandrum | 5.10⁶ per 1 ml |
| M1 microorganism | |

### Properties:

Stability: 6 months at a temperature of up to 25° C.

### Example 14

### Liquid Biological Antifungal Product for Treating Honeycombs

The product is designed for the preventative treatment of honeycombs from attack by a wide range of funguses, in particular of the Aspergillus and Fusarium families. The product can be used to treat combs intended for storage outside bee hives and combs within a bee hive not occupied by bees.

### Preparation:

Pythium oligandrum M1 biomass obtained by liquid cultivation was mixed in the vessel of a homogenizer (industrial mixer) with osmolyte (sucrose) and homogenized in such a way that the required number of oospores and concentration of osmolyte (sucrose) were achieved. Homogenization proceeded at 20,000 revolutions per minute for a period of 3 minutes. The suspension was then stored in sterile, stainless steel tanks at a temperature of under 8° C. The number of dormant oospores was determined microscopically with the use of a Sedgewick-Rafter counting chamber and stability was measured based on microscopic observation of the number of viable oospores using the plasmolytic method, in accordance with the publication by Etxeberria et al [13] from 2011. If the number of viable oospores did not fall below 90% of the initial value, the product was marked as stable.

### Composition:

| | | |
|---|---|---|
| Sucrose as stabilizer | 64% | weight |
| Water as stabilizer | 35.9% | weight |
| Culturing biomass | 0.1% | weight |

| | |
|---|---|
| Number of dormant oospores of the Pythium | 1.10⁶ per 1 ml |
| oligandrum M1 microorganism | |

### Properties:

Stability: 6 months at a temperature of up to 25° C.

### Example 15

### Liquid Biological Antifungal Product as an Anhydrous Suspension Concentrate Intended for Spraying Crops

Biomass obtained through cultivation of the Pythium oligandrum M1 microorganism on a solid substrate was mixed in a homogenizer vessel (industrial mixer) with demineralized water and homogenized. Homogenization proceeded at 3,000-5,000 revolutions per minute for a period of 3 minutes. After homogenization, the suspension obtained was mixed with an inorganic carrier (Sipernat), dried and ground in a cutting mill to achieve a resultant particle size of up to 350 μm. The dry culturing biomass with carrier was then mixed with paraffin oil so as to achieve the required concentration of oospores in the product. The suspension was stored in sterile containers at a temperature of up to 25° C.

The advantage of this product over example one is the longer stability of the product, which is ensured by the presence of water. The inorganic carrier, on a base of silicon oxide, ensures good suspensibility of solid particles (biomass) in the product.

The number of dormant oospores was determined microscopically with the use of a Sedgewick-Rafter counting chamber and stability was determined based on microscopic observation of the number of viable oospores using the plasmolytic method, in accordance with the publication by Etxeberria et al [13] from 2011.

### Composition:

| | | |
|---|---|---|
| Culturing biomass | 1.5% | weight |
| Sipernat 22 as inorganic carrier | 6.5% | weight |
| Paraffin oil as stabilizer | 92.0% | weight |

| | |
|---|---|
| Number of dormant oospores of the Pythium | 1.10⁶ per 1 ml |
| oligandrum M1 microorganism | |

### Properties:

Stability: 24 months at a temperature of up to 25° C.

### Example 16

### Liquid Biological Antifungal Product as an Anhydrous Suspension Concentrate Intended for Spraying Crops in the Organic Production of Agriculture Products

Biomass obtained through cultivation of the Pythium oligandrum M1 microorganism on a solid substrate was mixed in a homogenizer vessel (industrial mixer) with demineralized water and homogenized. Homogenization proceeded at 3,000-5,000 revolutions per minute for a period of 3 minutes. After homogenization, the suspension obtained was mixed with an inorganic carrier (Sipernat), dried and ground in a cutting mill to achieve a resultant particle size of up to 350 μm. The dry culturing biomass with carrier was then mixed with sunflower oil so as to achieve the required concentration of oospores in the product. The suspension was stored in sterile containers at a temperature of up to 25° C.

The advantage of this product over example one is the longer stability of the product, which is ensured by the presence of water. The inorganic carrier, on a base of silicon oxide, ensures good suspensibility of solid particles (biomass) in the product.

The number of dormant oospores was determined microscopically with the use of a Sedgewick-Rafter counting chamber and stability was determined based on microscopic observation of the number of viable oospores using the plasmolytic method, in accordance with the publication by Etxeberria et al [13] from 2011.

### Composition:

| | |
|---|---|
| Culturing biomass | 1.5% weight |
| Sipernat 22 as inorganic carrier | 6.5% weight |
| Sunflower oil as stabilizer | 92.0% weight |
| Number of dormant oospores of the Pythium oligandrum | 1.10⁶ per 1 ml |
| M1 microorganism | |

### Properties:

Stability: 24 months at a temperature of up to 25° C.

### Example 17

Liquid Biological Antifungal Product with Reduced Concentration of Oospores

Pythium oligandrum M1 biomass obtained by liquid cultivation was mixed in the vessel of a homogenizer (industrial mixer) with osmolyte (sucrose) and homogenized in such a way that the required number of oospores and concentration of osmolyte (sucrose) were achieved. Homogenization proceeded at 20,000 revolutions per minute for a period of 3 minutes. The suspension was thereafter mixed with osmolyte (65% sucrose) such that the concentration of oospores in the product matched the requirements of the product. The suspension was then stored in sterile, stainless steel tanks at a temperature of under 8° C.

The number of dormant oospores was determined microscopically with the use of a Sedgewick-Rafter counting chamber and stability was measured based on microscopic observation of the number of viable oospores using the plasmolytic method, in accordance with the publication by Etxeberria et al [13] from 2011. The number of dormant oospores is stabilized in the standard way if required.

### Composition:

| | | |
|---|---|---|
| Sucrose as stabilizer | 64.95% | weight |
| Water as stabilizer | 35.0% | weight |
| Culturing biomass | 0.05% | weight |

| | |
|---|---|
| Number of dormant oospores of the Pythium | 1 × 10³ per 1 ml |
| oligandrum M1 microorganism | |

### Properties:

Stability: 12 months at temperatures of from 2 to 8° C.; 6 months at a temperature of up to 25° C.

### Example 18

### Laboratory Test of the Effectiveness of Liquid Biological Antifungal Products in Mycoparasitic Experiments

A laboratory test of the proliferation of Pythium oligandrum M1 from dormant oospores of a suspension concentrate according to exemplary embodiment 1 and of subsequent mycoparasitism on the phytopathogenic fungus Fusarium graminearum was carried out on sterile agar plates with nutrient medium on a base of malt extract. The diluted concentrate was applied to an agar plate at application level, meaning 0.2 ml of concentrate per 1 liter of water, and part of this plate was removed after sprouting and transferred to a different agar, which was simultaneously inoculated with Fusarium graminearum fungus.

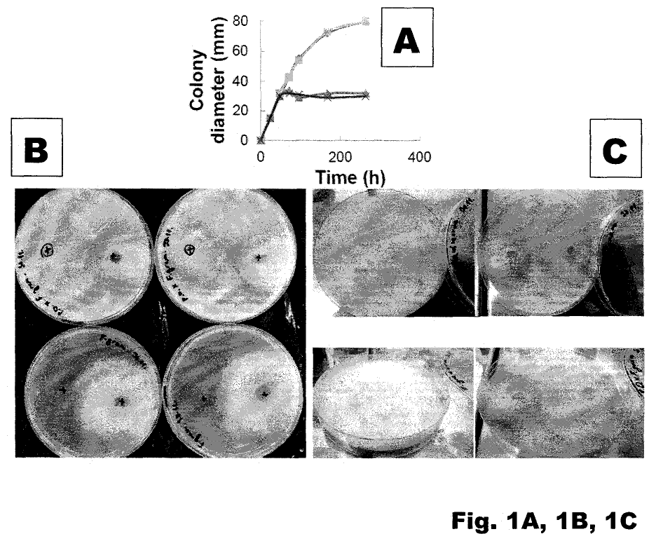

The result of this test is presented in FIG. 1, which shows a significant discontinuation of the growth of the phytopathogenic fungus Fusarium graminearum after the addition of the Pythium oligandrum M1 microorganism in comparison with standard growth (FIG. 1A). This situation is further illustrated using a series of Petri dishes photographed after 4 days, showing the joint growth of both microorganisms on the one hand and the growth of the phytopathogenic fungus on the other (FIG. 1B). After 11 days of culturing, significant suppression of the phytopathogenic fungus is clear alongside the growth of the Pythium oligandrum M1 microorganism. if the number of viable oospores did not fall below 90% of the initial value, the product was marked as stable.

### Example 19

Antifungal effectiveness against phytopathogenic funguses demonstrated in a laboratory experiment with wheat

Crop (wheat) seeds were treated with a solution of SAVO—sodium chlorite solution—(1-5% solution) and sprouted on agar soil extract. After sprouting, the shoots were again treated with 1% Sava solution and placed on a layer of phyto agar in a 250 ml Erlenmeyer flask. Flasks were inoculated with a piece of agar (5 mm) with the phytopathogen Fusarium graminearum or with the Pythium oligandrum M1 microorganism (dormant oospores in suspension concentrate) of application concentration of 0.2 ml per liter and volume of 0.2 ml in the following versions: 1) phytopathogen; 2) phytopathogen+Pythium oligandrum M1; 3) only Pythium oligandrum M1: control sample which was not inoculated. Flasks were sealed with a thin layer of cellulose and gauze. Cultivation proceeded in a room at a temperature of 24° C. The growth of the colony of phytopathogens and the Pythium oligandrum M1 microorganism was monitored, as was the condition of the plant.

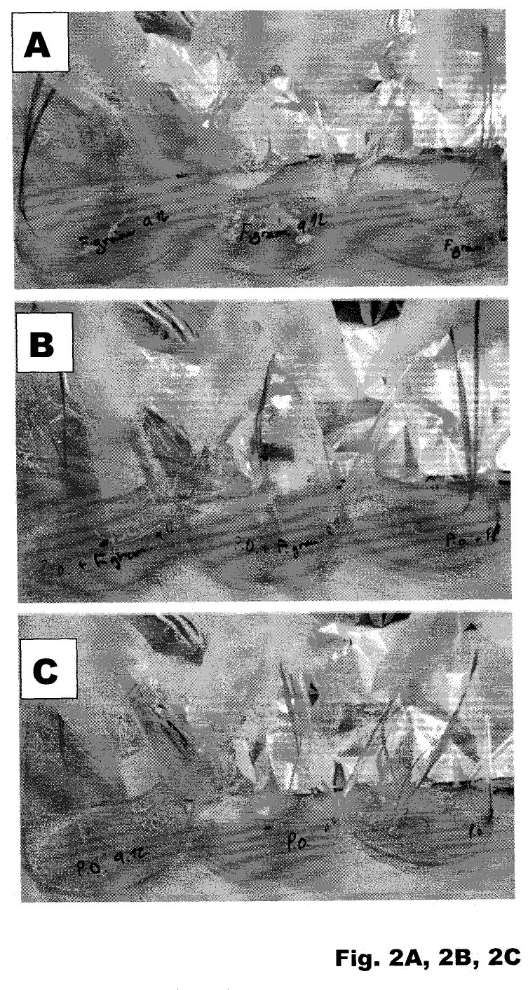

The result of this laboratory experiment is presented in FIG. 2. As far as the addition of the actual phytopathogen is concerned, its significant growth in the surroundings of the growing wheat is quite clear, as is the damage to the plant caused by the pathogen (FIG. 2A). If the Pythium oligandrum M1 microorganism was also added, in addition to the phytopathogenic fungus, the presence of the phytopathogen was not visible and the cultivated plant grew well (FIG. 2B). A control experiment carried out in the presence of the Pythium oligandrum M1 microorganism alone showed normal growth of the wheat plant which was comparable with experiments without the addition of any microorganism (FIG. 2C).

### Example 20

Test of the Effectiveness of a Liquid Biological Antifungal Product Intended for Spraying on Crops According to Exemplary Embodiment 2 in a Greenhouse Experiment on Brassica napus Rapeseed

Four sectors were used at the company greenhouse in Uherce containing growing Brassica napus rapeseed, Lohana C1 variety, whereby application was commenced at BBCh 15-21. Loose biofungicide with mycoparasitic microorganism Pythium oligandrum M1 (Polyversum®) was applied in the first sector, a new liquid fungicide according to exemplary embodiment 1 of this invention in sector 2, a new liquid antifungal product according to exemplary embodiment 2 of this invention in sector 3 and a new liquid anti-fungal product according to exemplary embodiment 4 of this invention in sector four. The application concentration in all cases was 0.5 g per liter, or 0.5 ml per liter, with dispersion of 100 ml of such concentrated product per 1 square meter of treated area. A collection point was marked in each sector for the collection of samples encompassing the plant and the soil between the rows. Photographic documentation and a reading of the temperature in the greenhouse at the time of collection were also taken during each collection. The collected samples were transferred to the laboratory within an hour and stored in a freezer at −20° C. until the time of analysis. Samples were collected before the application of the antifungal products and then 1 hour, 2 hours, 7 hours, 20 hours, 2 days, 3 days, 4 days, 5 days, 6 days, 7 days, 8 days, 11 days, 14 days, 17 days and 20 days after application.

The laboratory processing of samples involved photographic documentation of individual plants, the separation of leaf and root, determination of the wet weight of the sample, homogenization of the sample in DNA extraction buffer, centrifugation and the conduction of molecular analyses. The quantitative determination of the presence of the Pythium oligandrum M1 microorganism was carried out using the qPCR method with a probe targeted at the ITS sequence, while the quantitative determination of the DNA of rapeseed was conducted using an identical method with a probe targeted at the Fat(A)A sequence. The quantitative determination included the execution of 50 cycles of DNA amplification under standard conditions and the calculation of the concentration of target sequences using calibration curve. The level of fungal infestation was detected in a similar way based on standard protocol.

The results of the effectiveness test are presented in Table 1 below. The effectiveness achieved by the new liquid antifungal products according to this invention is in all cases comparable with or better than the effectiveness of the solid, loose product. Foreign funguses were always suppressed depending on the colonization and amplification of the Pythium oligandrum M1 microorganism in the place of application. The low level of contamination by funguses was thereafter maintained even after reducing the concentration of the Pythium oligandrum M1 microorganism at the end of the experiment. The dynamic of the population of the Pythium oligandrum M1 microorganism was monitored in this experiment only for a period of 15 days, resulting in the absence of any comment on long-term effects. Nonetheless, from the perspective of the antifungal effectiveness of the product there is no doubt that the ability to suppress fungus is significant and long-term: there was a reduction in the occurrence of such funguses of approximately one order 24 hours after application and a further reduction of almost one order after a further 24 hours.

TABLE 1
Measurement of the content of the Pythium oligandrum M1 microorganism
and measurement of the content of fungus on the leaves of rapeseed
in a greenhouse experiment. The results are shown as the numbers
of detected diploid genomes per 1 □g of host DNA of rapeseed.

| Time of | | | | | | | | |
|---|---|---|---|---|---|---|---|---|
| collection | Sector 1 | | Sector 2 | | Sector 3 | | Sector 4 | |
| (h) | Pythium | Fungus | Pythium | Fungus | Pythium | Fungus | Pythium | Fungus |
| 0 | 8 × 10² | 1 × 10⁶ | 7 × 10² | 1 × 10⁶ | 8 × 10² | 2 × 10⁶ | 6 × 10² | 2 × 10⁶ |
| 2 | 2 × 10⁴ | 8 × 10⁵ | 1 × 10⁴ | 1 × 10⁶ | 1 × 10⁴ | 1 × 10⁶ | 2 × 10⁴ | 1 × 10⁶ |
| 20 | 2 × 10⁵ | 1 × 10⁵ | 2 × 10⁵ | 2 × 10⁵ | 2 × 10⁵ | 3 × 10⁵ | 2 × 10⁵ | 2 × 10⁵ |
| 26 | 5 × 10⁵ | 6 × 10⁴ | 6 × 10⁵ | 6 × 10⁴ | 7 × 10⁴ | 5 × 10⁴ | 6 × 10⁵ | 5 × 10⁴ |
| 44 | 1 × 10⁶ | 2 × 10⁴ | 2 × 10⁶ | 2 × 10⁴ | 1 × 10⁶ | 2 × 10⁴ | 2 × 10⁶ | 1 × 10⁴ |
| 92 | 8 × 10⁵ | 1 × 10³ | 8 × 10⁵ | 2 × 10³ | 7 × 10⁵ | 1 × 10³ | 7 × 10⁵ | 3 × 10³ |
| 140 | 1 × 10⁴ | 5 × 10² | 3 × 10⁴ | 6 × 10² | 4 × 10⁴ | 6 × 10² | 4 × 10⁴ | 5 × 10² |
| 212 | 1 × 10⁴ | 5 × 10² | 2 × 10⁴ | 5 × 10² | 2 × 10⁴ | 7 × 10² | 2 × 10⁴ | 6 × 10² |
| 356 | 8 × 10³ | 6 × 10² | 1 × 10⁴ | 4 × 10² | 1 × 10⁴ | 5 × 10² | 1 × 10⁴ | 4 × 10² |

### Example 21

Test of the Effectiveness of a Liquid Biological Antifungal Product Intended for Spraying on Crops According to Exemplary Embodiment 1 in a Field Test on Triticum aestivum Wheat

The winter wheat Triticum aestivum was sown in October 2015 in the company field close to the village of Úherce u Loun. The antifungal product according to exemplary embodiment 1 of this invention was applied to the designated tracks in November 2015 using the standard spraying method in a quantity of 100 ml per hectare in accordance with agricultural standards and approved registration for the Polyversum® antifungal preparation for plant protection. Samples were taken from 4 designated collection points before the application and at different numbers of days after application, whereby the growing wheat plants and the soil in proximity to the sample were collected. The samples were frozen immediately after collection and stored in sealed bags or test tubes at −20° C. until the time of analysis. Nucleic acids were extracted from the samples collected using a published method (Klimeš R, Suchánek M, Maštalková L et al/2016/Comparison of the efficacy of treatment of dermatophytosis by chemical and biological antifungals: soil peronosporomycete Pythium oligandrum is as efficient as the antifungal enilconazole in the guinea pig model, Vet Dermatol, in print) and used for molecular analysis).

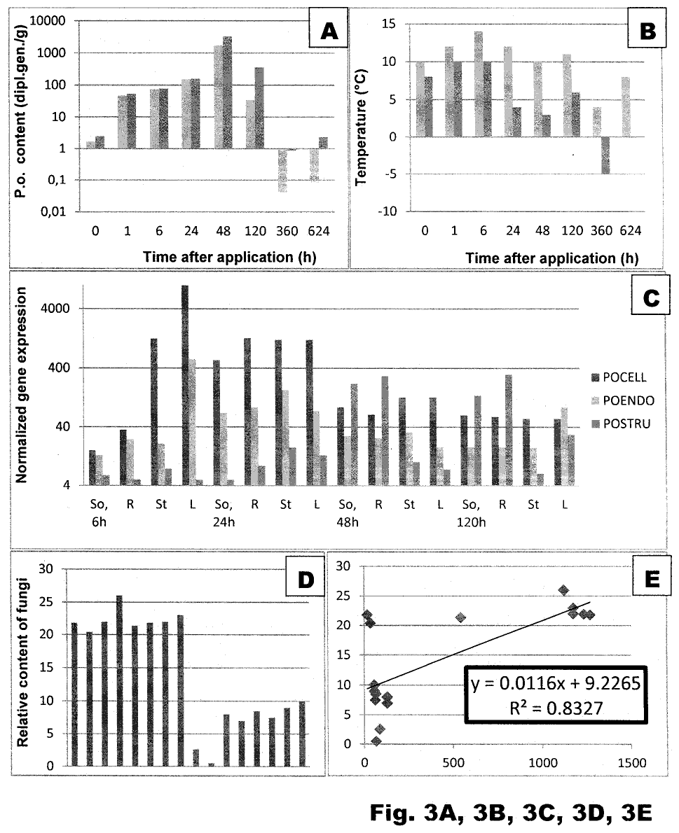

As far as the dynamic of the occurrence of the Pythium oligandrum M1 microorganism following spraying with the liquid product according to exemplary embodiment 1 is concerned, the standardized concentrations on plants and in the surrounding soil showed similar dependence. Whereas only individual cells per g of sample could be identified by qPCR genetic test prior to the application, this number rose to hundreds of cells per g of sample following the application of the liquid product corresponding to the normal standardized quantity. There was visible multiplication of the Pythium oligandrum M1 microorganism under the experimental conditions in place after 48 hours, whereby this positive trend was interrupted under the conditions of a specific experiment by a frost episode at the beginning of December 2015, after which the dynamic population growth was not restored (compare FIG. 3A and FIG. 3B).

A quantity of valuable information was obtained through an analysis of gene expression profiles using the RT-qPCR method conducted at the “peak” phase of the experiment, with the highest growth of the Pythium oligandrum M1 microorganism. The values of the concentration of transcripts showed fast reaction to the occurring fungal pathogens at the first phase (significant expression of gene for cellulase) followed by an increased occurrence of zoospores (endoglucanase expression). Only after 48 hours and 120 hours is the characteristic gene marker of sporulation heightened, primarily in the surrounding soil and roots (FIG. 3C). At the same time, it was possible to observe a significant reduction in the relative content of fungus 48 hours after application (FIG. 3D) and this reduction reciprocally correlated with the expression of the gene for cellulose responsible for the degradation of surface saccharides in funguses during the mycoparasitic action of the Pythium oligandrum M1 microorganism (FIG. 3E).

### Example 22

Effectiveness Test of Toothpastes in the Oral Biofilm Model with Mechanical Plaque Removal

A laboratory test using the oral biofilm model with mechanical removal of plaque was conducted according to the publication of Verkaik M J et al [14] from 2010 in order to monitor the effectiveness of prepared toothpastes. A version (B) of the above-published protocol was used, consisting of two-hour adhesion with subsequent growth overnight (16-hour biofilm) and mechanical cleaning. The effectiveness of glycerol toothpaste containing the Pythium oligandrum M1 microorganism and toothpastes with olive oil and the Pythium oligandrum M1 microorganism in removing the microorganisms of dental plaque was compared with two types of commonly-available toothpastes (Odol and Enzycal) and, as a control, mechanical cleaning with a toothbrush without any paste (marked Control). Identical paste without any Pythium oligandrum content and without the use of silicon oxide as an abrasive was used in a control experiment.

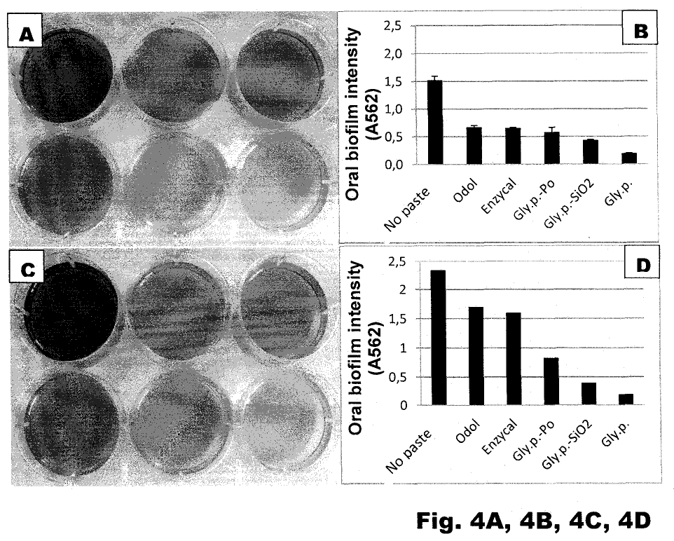

The outcome of this test is described in the table of results in FIG. 2. This showed that paste according to exemplary embodiment 4 exhibited considerably higher effectiveness in the removal of dental biofilm and plaque than the alternative products used, even among individuals affected by periodontal disease (FIG. 4A to FIG. 4D). Whereas the pastes commonly used until now reduced the intensity of biofilm formation to around one half, the new pastes according to this invention to up to one tenth. Its effect in the control paste without any Pythium oligandrum M1 microorganism content was comparable with common pastes, while the effect was significantly higher in the control paste without silicon oxide. The overall level of biofilm formation in the patient with periodontal disease was significantly higher and it was also far better to observe the differences in the effectiveness of individual pastes.

### Example 23

Test of the Effectiveness of the Liquid Biological Antifungal Product According to Exemplary Embodiment 1 in Protecting Living Spaces from Fungal Spores

The antifungal product according to exemplary embodiment 1 was diluted with water to a concentration of 330,000 oospores per liter of application solution, i.e. 0.66 ml of liquid concentrate was added to 1 liter of solution. A control solution was also prepared under identical conditions, but without Pythium oligandrum M1 microorganism content; the biomass was replaced with water. The solutions prepared in this way were applied in two small, enclosed rooms at the farmstead at Úherce u Loun No. 2 in the form of spraying a fine mist using an atomizer. Following application, both rooms were impermeably closed and fallout tests were conducted on the content of spores using the Omeljanský method. The results were evaluated in the laboratory of Dr. Miroslav Kolařík at the Institute of Microbiology of the Academy of Sciences of the Czech Republic in Prague.

The results of the experiment are presented below in Table 2. The experiment was conducted in closed rooms without any flow of air and for this reason the observed changes are slower and were observed only after several months. Nonetheless, it is clear from the results that in control room 1, with the application of auxiliary substances, the numbers of spores identified fluctuated, but did not at any time of observation fall below the WHO (World Health Organization limit) of 500 spores per 1 cubic meter of air. By contrast, there was a statistically significant decrease in spores 3 months after application in room 2 and this subsequently fell below the WHO limit 6 months following the date of application.

TABLE 2
Measurement of the number of fungal spores per 1 cubic
meter in room 1, with the application of a control product,
and in room 2, with the application of a product containing
the active ingredient Pythium oligandrum M1

Months after application (0 -
before application)

| | 0 | 1 | 3 | 6 |
|---|---|---|---|---|
| Room 1, control | 5000 | 4500 | 3500 | 4500 |
| Room 2 | 5000 | 4000 | 1300 | 400 |
| (product accord, | | | | |
| to exemplary | | | | |
| embodiment 1) | | | | |

### Example 24

Method of Production of a Liquid Biological Antifungal Product Containing Pythium oligandrum M1

The detailed nature of the liquid biological antifungal product containing Pythium oligandrum M1 according to this invention was demonstrated in detail in the previous exemplary embodiments.

Examples of the method of production of the liquid biological antifungal product containing Pythium oligandrum M1 according to this invention are presented below.

A medium containing extract of cereals, cane molasses and other essential nutrients is used for the aerobic cultivation of Pythium oligandrum M1 oomycetes. The liquid medium is sterilized in a steam sterilizer. After cooling, it is inoculated with one of the selected strains of Pythium oligandrum M1. After cultivation has ended, a process which takes approximately 13 days, the biomass is harvested and processed in the manner presented above in the exemplary embodiments.

A medium is prepared for cultivation the Pythium oligandrum M1 microorganism on a solid substrate containing cereal grains, preferably hulled millet grains, for example Panicum miliaceum L., which are mixed with a percentage of nutrient liquid medium. The medium is sterilized in cultivation vessels in a steam sterilizer. After cooling, the medium is inoculated with one of the selected strains of Pythium oligandrum M1. After the process of cultivation has ended, a process which takes approximately 8 days, the harvested biomass is further processed into the form of a suspension concentrate, as is described above in the exemplary embodiments.

After the end of the culturing stage of Pythium oligandrum in the liquid phase and in solid phase, the biomass is homogenized with the liquid medium, for example in an industrial mixer, so that a minimum of 95% of the particles in the suspension are between 0.05 and 0.30 mm, preferentially between 0.050 and 0.125 mm. The homogenous suspension is subsequently characterized by the number of oospores. The suspension is standardized—concentrated or diluted—according to the number of oospores to the pre-determined concentration of oospores suitable for the preparation of the liquid biological antifungal product at issue.

After the end of the culturing phase of Pythium oligandrum M1 on a solid substrate, the biomass is homogenized, preferentially with an industrial mixer, in the corresponding volume of demineralized water so that the resulting size of a minimum of 95% of the particles in the suspension is between 0.050 and 0.300 mm in size, preferentially between 0.050 and 0.125 mm. The homogenous suspension is subsequently characterized by the number of oospores. The suspension obtained is concentrated or diluted according to the number of oospores to the pre-determined concentration of oospores suitable for the preparation of the liquid biological antifungal product at issue.

The suspension is separated, for example filtered, after the phase of homogenization and concentration, or dilution, to ensure the maximum admissible size of particles within the relevant range. If achieving particles of around 0.05 mm to 0.125 mm, there is a high probability of obtaining a concentrated suspension, or an almost pure concentrated suspension of oospores of Pythium oligandrum M1. Most of the exemplary embodiments presented above had a size of particles of predominantly 0.050 to 0.125 mm.

An aqueous suspension may be stabilized during homogenization with an additive of osmolyte and can be stored in large, sterile containers at temperatures of lower than 8° C.

In the case of an anhydrous suspension of the Pythium oligandrum M1 microorganism, the material obtained after the end of the culturing stage on a solid substrate or in liquid phase is homogenized, preferentially with an industrial mixer, so that the resulting size of a minimum of 95% of the particles in the suspension is between 0.050 and 0.300 mm, preferentially between 0.050 and 0.125 mm. The homogenous suspension obtained, characterized by the number of oospores, is subsequently centrifuged and, after removal of the supernatant, oil is added to the centrifuged material to achieve the pre-determined concentration of oospores, as appropriate for the preparation of the liquid biological antifungal product at issue. The material obtained is subsequently re-suspended, for example using an industrial mixer.

Specific possible methods of production are specified in the exemplary embodiments presented above, but are not limited to these.

### INDUSTRIAL APPLICABILITY

The new liquid antifungal product containing a stabilized suspension of oospores can be made in sizeable volumes mainly because the fermentation production phase and the formulation stage are both well manageable from the technical perspective. The new liquid product can find an application in all areas in which other forms of microscopic oomycetes of Pythium oligandrum have been applied and used until now, in particular in the biological protection of plants, to protect buildings and residences from funguses, to suppress funguses and yeasts and to establish physiological balance of microflora in human and veterinary applications. Its use as a protective spray for plants with degradable polymer, acting as a protective factor and as a slow-release, degradable matrix “carrier” of the Pythium oligandrum microorganism, is highly innovative. The product according to this invention can also be beneficially used to coat or spray wooden structures, when the oil components of the product also lead to effective impregnation of the wood. The presence of the microorganism guarantees resistance to funguses and fungal diseases and “defects” to wood. One entirely new use is the effective conservation of air-treatment equipment, air-conditioning and cooling units. It would also appear advantageous to use a product specially designed for purpose to reduce the level of fungal contamination in areas affected by flooding, in dried-out areas and in the surroundings of recultivated areas of water.

1. A liquid biological antifungal product including
a stabilized suspension of a Pythium oligandrum microorganism, the liquid biological antifungal product comprising:
0.05 to 10.0% weight culturing biomass of the Pythium oligandrum microorganism, including
cultivation medium, cell forms of the Pythium oligandrum microorganism and substances produced by the Pythium oligandrum microorganism;
and
90.0 to 99.95% weight stabilizer;
wherein a pre-determined number of dormant oospores in 1 ml of the liquid biological antifungal product is, following normal standardization, between 1×10³ and 2×10⁷.

2. (canceled)

3. The liquid biological antifungal product according to claim 1, wherein
the Pythium oligandrum microorganism is comprises the Pythium oligandrum Dreschler ATTC 38472 strain.

4. The liquid biological antifungal product according to claim 1, wherein the weight stabilizer contains at least one component from a group including water, a salt solution, an oil or an osmolyte solution.

5. The liquid biological antifungal product according to claim 4 wherein
the stabilizer is water, and wherein the stabilizer is between 30.0 and 99.9% weight.

6. The liquid biological antifungal product according to claim 4 wherein
the stabilizer is the salt solution, and wherein the stabilizer is 99.9% weight.

7. The liquid biological antifungal product according to claim 4 wherein
the stabilizer is the osmolyte, wherein the osmolyte is in the form of sucrose, and wherein the stabilizer is between 60.0 and 64.95%
weight.

8. The liquid biological antifungal product according to claim 4 wherein
the stabilizer is the oil, and wherein the oil comprises at least one of the following: paraffin oil,
mineral oil, glycerol or sunflower oil in a quantity of 79.77 to 99.9% weight.

9. The liquid biological antifungal product according to claim 4 wherein the stabilizer is the osmolyte, wherein the osmolyte is glycerol, and wherein the osmolyte is between 79.77 and 99.9% weight.

10.-11. (canceled)

12. A method of producing the liquid biological antifungal product
according to claim 1, comprising:
a) sterilizing a liquid medium comprising extract of cereals, cane molasses, and other essential nutrients to aerobically culture oomycetes of Pythium oligandrum in liquid phase;
b) inoculating the liquid medium with
Pythium oligandrum;
c) harvesting a biomass;
d) homogenizing the biomass with the liquid medium so that a minimum of 95% of the particles in the suspension are between 0.050 and 0.300 mm, preferentially between 0.050 and 0.125 mm;
e) standardizing the homogenized suspension according to the pre-determined number of dormant oospores in 1 ml of the liquid biological antifungal product.

13. The method of producing the liquid biological antifungal product
according to claim 12, wherein
the extract of cereals comprises hulled millet grains, including Panicum miliaceum L.

14. The method of producing the liquid biological antifungal product according to claim 12, further comprising
centrifuging the homogenized suspension obtained
and, after removal of a supernatant, adding oil to the centrifuged material to achieve the pre-determined number of dormant oospores in 1 ml of the liquid biological antifungal product; and
re-suspending the material.

15. The method of producing the liquid biological antifungal product according to claim 14, further comprising filtering the suspension
to the pre-determined size of particles in order to ensure the maximum admissible size of particles of the Pythium oligandrum microorganism of between 0.050 and 0.300 mm, preferentially 0.050 to 0.125 mm.

16. The method of producing the liquid biological antifungal product according to claim 12, wherein standardizing the homogenized suspension comprises adding an osmolyte and storing the suspension at temperatures of lower than 8° C.

17. A liquid biological antifungal product including a stabilized suspension of the Pythium oligandrum microorganism, the liquid biological antifungal product comprising:
0.05 to 10.0% weight culturing biomass of a Pythium oligandrum microorganism, including cultivation medium, cell forms of the Pythium oligandrum microorganism, and substances produced by the Pythium oligandrum microorganism;
79.77 to 99.95% weight stabilizer;
a remaining up to 100% weight including at least of one of the following
modifying/application
substances: a filling, an aroma and vitamin E;
wherein a pre-determined number of dormant oospores in 1 ml of the liquid biological antifungal product is, following normal standardization, between 2.5×10⁴ and 1.0×10⁶.

18. The liquid biological antifungal product according to claim 17, further comprising 16.0% weight slow-release biodegradable matrix as a carrier of oospores of the Pythium oligandrum microorganism.

19. The liquid biological antifungal product according to claim 18, wherein the slow-release biodegradable matrix is polyvinyl alcohol.

20. The liquid biological antifungal product according to claim 17, further comprising 16.0% weight filling based on silicon oxide.

21. The liquid biological antifungal product according to claim 17, further comprising an application/modifying substance comprising 0.96% weight sage or mint aroma and 0.4% weight vitamin E.
